(19) United States
(12) Patent Application Publication
(10) Pub. No.: US 2016/0270649 A1
(43) Pub. Date: Sep. 22, 2016

(54) VITAL STAIN VISUALIZATION IN OPHTHALMIC SURGICAL PROCEDURES AND ASSOCIATED DEVICES, SYSTEMS, AND METHODS

(71) Applicant: NOVARTIS AG, Basel (CH)

(72) Inventors: Steven T. Charles, Germantown, TN (US); Michael Papac, Lake Forest, CA (US)

(21) Appl. No.: 15/168,946

(22) Filed: May 31, 2016

Related U.S. Application Data
(63) Continuation of application No. 14/446,586, filed on Jul. 30, 2014.

(51) Int. Cl. A61B 3/00; A61B 3/14; A61B 3/13; A61F 9/007

(52) U.S. Cl. CPC A61B 3/0025; A61F 9/00736; A61B 3/14; A61B 3/0041; A61B 3/13

(57) ABSTRACT

A method of imaging in an ophthalmic surgical procedure can include determining an excitation wavelength of light associated with a vital stain; transmitting light having the excitation wavelength; determining an emission wavelength of light associated with the vital stain; filtering light using a first optical element to allow transmission of light having the emission wavelength and to block light having the excitation wavelength. An ophthalmic surgical imaging system can include a light source, one or more optical elements, an image sensor, a computing device, and/or a display device to visualize target biological tissue stained with a fluorescent vital stain. A method of imaging in an ophthalmic surgical procedure can include determining a wavelength of light that increases the visual contrast of a vital stain; transmitting light having the determined wavelength; and receiving a reflection of the transmitted light such that target biological tissue stained by the vital stain is accentuated.

### BACKGROUND

1. Technical Field

Embodiments disclosed herein are related to ophthalmic surgical imaging. More specifically, embodiments described herein relate to fluorescence imaging with controlled excitation of a vital stain and collection of emitted fluorescent light during a surgical procedure.

2. Related Art

Ophthalmic surgical procedures can involve the manipulation of extremely thin and transparent tissue in the eye, including the lens, cornea, and retina, among other tissue. For example, the retina, the lens capsule, and the inner limiting membrane (ILM), which are important surgical targets, are respectively 200 microns, 100 microns, and 3 microns thick. A surgeon must be able to readily identify the thin, transparent tissue in order to safely grasp it using an instrument during the surgical procedure. For example, peeling the ILM is a common surgical approach in macular surgery. It is also one of the most difficult surgical maneuvers to accomplish due to the close proximity of instruments to the macula and the resulting dexterity required of the surgeon.

Vital stains can be applied to the eye to assist in visualizing one or more surgical targets. For example, an ILM stained with indocyanine green (ICG) can be more readily visualized than if it were not. Several factors can be considered when a stain is selected to be applied in the eye, including toxicity to the retina, visibility of stained tissue, affinity to tissue upon application, chemical characteristics (e.g., stability, concentration, pH, etc.), among other factors. ICG, for example, while effective in staining the ILM, has been shown to be toxic. Other less toxic stains can be associated with low visibility and/or low affinity, and thus are passed over when considered for use. This can result in increased health risks to patients.

Other health risks for patients can exist without the ability to readily visualize surgical targets. For example, panretinal photocoagulation (PRP) can be used to treat sites in the peripheral retina expressing factors that drive neovascularization such as vascular endothelial growth factor (VEGF). Conventionally, expression sites cannot be directly visualized. Thus, the surgeon must make thousands of retina burn spots in order to achieve sufficient coverage to destroy expression sites. Large areas of burn spots can damage peripheral vision and may not be effective in treating the disease. Other complications also arise when too strong or too weak burns are used.

Ophthalmic surgical procedures have conventionally used endoilluminators with broadband white light (e.g., halogen, xenon, etc.) to illuminate the surgical field. Further, surgeons can rely merely on reflectance of light to visualize surgical targets (e.g., through a surgical microscope). The use of fluorescence in ophthalmic procedures has conventionally been limited to in-office studies of vasculature in the eye. For example, fluorescein dye and ICG have been used to identify abnormal retinal blood vessels. Electrons of a fluorescent material are excited when energy (e.g., light) is applied and absorbed. Once excited, the material emits light or fluoresces as the electrons return to their ground state. Fluorescence has not been used during ophthalmic surgical procedures to increase visualization of surgical targets.

### SUMMARY

The presented solution fills an unmet medical need with a unique solution to provide an ophthalmic surgical imaging system with optical elements to control the excitation of a fluorescent stain and collection of the emitted fluorescent light to visualize biological tissue. The efficient, controlled excitation and collection of fluorescent light can improve the surgeon's ability to resolve fine details of the patient's eye during the ophthalmic surgical procedure. The present disclosure also describes selectively illuminating a surgical field with wavelength(s) of light that increase the visual contrast of a vital stain such that target biological tissue is accentuated.

Consistent with some embodiments, a method of imaging in an ophthalmic surgical procedure can include: determining an excitation wavelength of light associated with a vital stain disposed in a surgical field; transmitting light having the excitation wavelength to the surgical field; determining an emission wavelength of light associated with the vital stain; filtering light from the surgical field using a first optical element to allow transmission of light having the emission wavelength and to block transmission of light having the excitation wavelength.

Consistent with some embodiments, an ophthalmic surgical imaging system can include: a light source controllable to transmit light having an excitation wavelength associated with a vital stain disposed in a surgical field; a first optical element disposed in an optical pathway of light transmitted from a surgical field, the first optical element being controllable to selectively filter light received from the surgical field to allow the transmission of light having an emission wavelength associated with the vital stain and to block the transmission of light having the excitation wavelength; an image sensor disposed in the optical pathway of light transmitted from the surgical field, the image sensor being configured to receive the filtered light having the emission wavelength from the first optical element; a computing device in communication with the image sensor and configured to process the received light to generate image data including modifying a characteristic of the image data to enhance visualization of the target biological tissue in a visual representation; and a display device in communication with computing device and configured to display the visual representation.

Consistent with some embodiments, a method of ophthalmic imaging can include: determining a wavelength of light that increases the visual contrast of a vital stain disposed in a procedure eye; transmitting light having the determined wavelength to the procedure eye including at least one of: controlling a tunable light source to transmit the light having the determined wavelength; and filtering light from a light source with an optical element positioned in an optical pathway of light transmitted by the light source to transmit the light having the determined wavelength; and receiving a reflection of the light transmitted to the procedure eye such that target biological tissue stained by the vital stain is accentuated compared to other portions of the procedure eye.

Additional aspects, features, and advantages of the present disclosure will become apparent from the following detailed description.

### BRIEF DESCRIPTION OF THE DRAWINGS

FIG. 1 is a flow diagram illustrating a method of imaging in an ophthalmic surgical procedure.

FIG. 2 is a diagram illustrating an ophthalmic imaging system.

FIG. 3 is a diagram illustrating an ophthalmic imaging system.

FIG. 4 is a diagram illustrating an ophthalmic surgical instrument.

In the drawings, elements having the same designation have the same or similar functions.

### DETAILED DESCRIPTION

In the following description specific details are set forth describing certain embodiments. It will be apparent, however, to one skilled in the art that the disclosed embodiments may be practiced without some or all of these specific details. The specific embodiments presented are meant to be illustrative, but not limiting. One skilled in the art may realize other material that, although not specifically described herein, is within the scope and spirit of this disclosure.

The present disclosure describes devices, system, and methods of controlled, narrow band illumination of a fluorescent vital stain used to visualize a target biological tissue during an ophthalmic surgical procedure. The illumination can be provided by a light source and an optical element. The fluorescent light can be collected in a controlled manner using an optical element. The fluorescent target biological tissue can be visualized directly using surgical microscope optics. The fluorescent light can also be received at an image sensor/camera. Image data can be processed to enhance the target biological tissue stained by the vital stain. The enhanced image data can be provided to a display device.

The devices, systems, and methods of the present disclosure provide numerous advantages, including: (1) improving efficacy of surgical procedures by providing improved visualization of surgical targets for the surgeon; (2) improving safety for the patient by allowing the use of less toxic vital stains with low affinity and/or low visibility; (3) improving safety for the patient by allowing use of lower doses of known stains to reduce toxicity; and (4) increasing utility for surgical microscopes by maximizing surgical field sight for all surgeons.

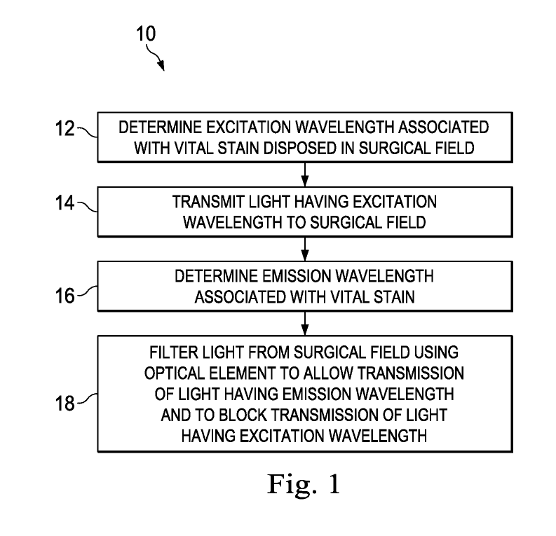

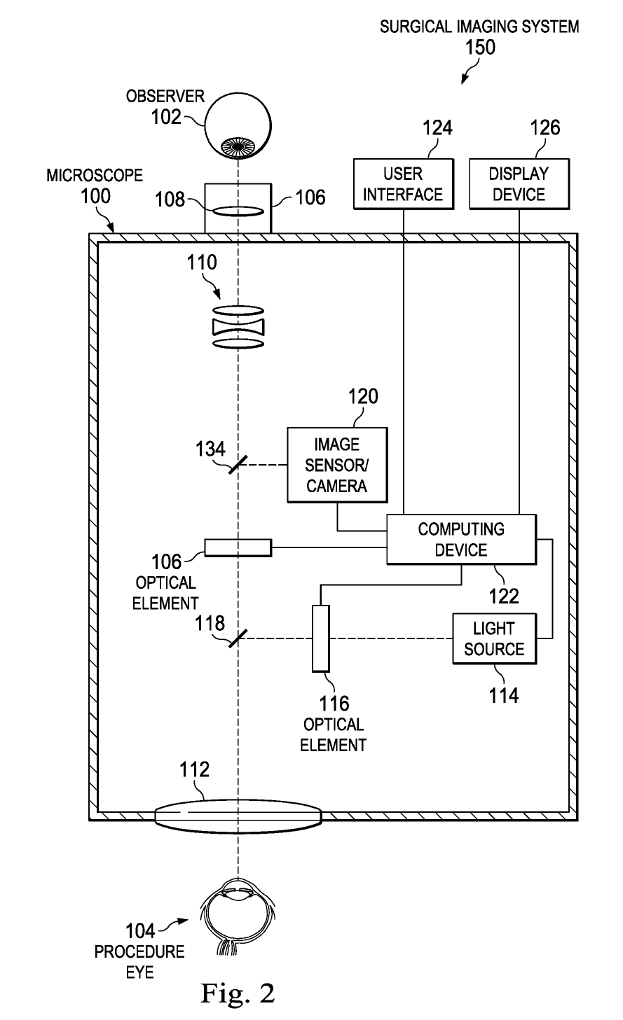

FIG. 1 provides a flow diagram of a method 10 of imaging in an ophthalmic surgical procedure. The method 10 can be further understood with reference to FIGS. 2-4. The method 10 can be implemented during a surgical procedure in which a vital stain is used to improve visualization of a surgical target. In some embodiments, the vital stain can be intravitreally injected or otherwise intravitreally administered to stain a target biological tissue in the procedure eye and/or the surgical field. In some embodiments, the vital stain can be intravenously introduced to a target biological tissue in the procedure eye and/or the surgical field. In some embodiments, the vital stain can be delivered to the surgical field such that the vital stain is infused into the procedure eye and/or the target biological tissue. The method 10 can include, at step 12, determining excitation wavelength(s) of light associated with the vital stain. The excitation wavelength of the vital stain can be used to efficiently illuminate the surgical field such that the vital stain fluoresces and increases the visibility of the target biological tissue. The excitation wavelength(s) can include a single wavelength, such as a peak excitation wavelength, or a range of wavelengths. The range of wavelengths can be centered on a particular wavelength (e.g., all wavelengths within +/−25 nm of the peak excitation wavelength). The span of the range can be fixed for all vital stains or variable depending on the particular vital stain used during the surgical procedure.

Determining the excitation wavelength(s) can include receiving a user input identifying the stain used during the ophthalmic surgical procedure. The user input can be received at a user interface 120, described in greater detail below. A computing device 122, also described in greater detail below, can determine the excitation wavelength(s) associated with the inputted vital stain. For example, the computing device 122 can access a database of vital stains and their corresponding excitation wavelength(s), and determine the excitation wavelength(s) of the inputted vital stain using the database. In some embodiments, a user input directly identifying the excitation wavelength(s) can be received at the user interface 120.

The method 10, at step 14, can include transmitting light having the excitation wavelength(s) to the surgical field. The surgical field can be illuminated with wavelengths of light from a light source 114 and/or an optical element 116 that correspond to the excitation wavelength(s) of the vital stain determined in step 12. Such illumination can allow the vital stain to be efficiently excited when the light transmitted to the surgical field is absorbed. As a result, the vital stain can fluoresce more efficiently, and the target biological tissue to which the stain has been applied can be better visualized.

The light source 114 can be configured to transmit light to excite the vital stain applied to the target biological tissue in the surgical field. In some embodiments, the light source 108 can be a tunable light source such that the transmitted wavelength(s) are selectable based on the excitation wavelength(s) determined in step 12. The light source 108 can include super-luminescent diodes, an ultra-short pulsed laser, a super-continuum laser, a titanium-sapphire laser, an incandescent light bulb, a halogen light bulb, a metal halide light bulb, a xenon light bulb, a mercury vapor light bulb, a light emitting diode (LED), etc. The light source 114 can be in communication with the computing device 122. The computing device 122 can provide a control signal to the light source 114 to transmit light to the surgical field based on the excitation wavelength(s).

The optical element 116 can be a spectral filtering element positioned in an optical pathway of the light transmitted by the light source 108. The optical element 116 can be configured to filter the light transmitted to the surgical field based on the excitation wavelength(s) of the vital stain. For example, the optical element 116 can block the transmission of light except for wavelength(s) corresponding to the excitation wavelength(s). The optical element 116 can be a dichroic filter, a diffraction grating, an opto-acoustic tunable filter (AOTF), a liquid crystal tunable filter (LCTF), a linear-variable filter (LVF), a micro-electro-mechanical systems (MEMS) tunable optical filter, interference filter, etc. The optical element 116 can be in communication with the computing device 122. The computing device 122 can provide a control signal to the optical element 116 to filter light based on the excitation wavelength(s).

Controlling the light source and/or the optical element (step 14) can include illuminating the surgical field with narrow bandwidth light (in contrast to broadband, white light). The emission band of light transmitted to the surgical field can be between 1 nm and 100 nm, 1 nm and 50 nm, between 1 nm and 25 nm, 1 nm and 20, and 1 nm and 10 nm, etc., though larger and smaller bandwidths are contemplated. The narrow bandwidth can correspond to the excitation wavelength(s) determined in step 12. In some embodiments, the emission band of the light source 114 is controllable such that the light source 114 transmits narrow bandwidth light. In some embodiments, the optical element 116 can be controlled to permit only narrow bandwidth light to pass therethrough.

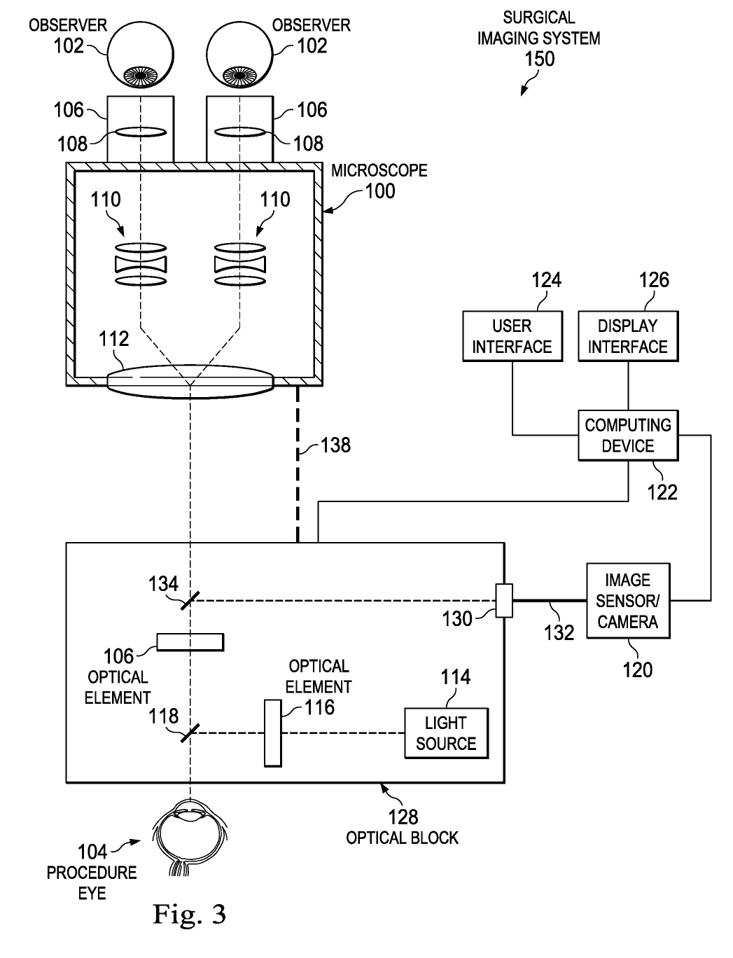

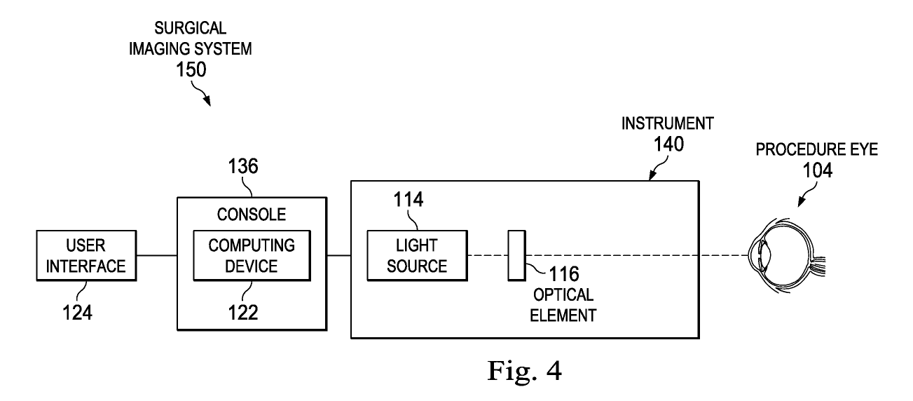

The light source 114 and/or the optical element 116 can be included in one or more components of a surgical imaging system 150 (FIGS. 2-4). For example, the light source 114 and/or the optical element 116 can be integrated in an ophthalmic surgical microscope 100 (FIG. 2), an optical block 128 that is distinct from but coupled to the microscope 100 (FIG. 3), and/or an ophthalmic surgical instrument 140 (FIG. 4).

FIGS. 2-3 illustrate the ophthalmic surgical microscope 100. An observer 102 can view the surgical field, such as a procedure eye 104, using the microscope 100. The observer 102 can be a healthcare professional, such as a doctor or surgeon performing a diagnostic, surgical, and/or other medical procedure. The procedure eye 104 can be that of a patient undergoing the medical procedure. The target biological tissue can be tissue in the procedure eye 104. One or more lenses, mirrors, filters, gratings, and/or other optical components can comprise an optical train of the microscope 100. The optical components can be positioned in the optical pathway of light transmitted from the surgical field. For example, eyepieces 106 can include optical components 108, and the body of the microscope 100 can include optical components 110 and objective lens 112. The optical components 108 and 110, and objective lens 112 are exemplary, and in various embodiments, the microscope 100 can include more or fewer lenses and/or other optical components to focus the light and/or magnify the image. The ophthalmic surgical microscope 100 can be positioned in an optical pathway of light transmitted from the surgical field.

In some embodiments, the light source 114 and/or the optical element 116 can be coupled to the microscope 100, directly or indirectly, such that the light source 114 and/or the optical element 116 have a defined optical/optomechanical relationship to the surgical microscope 100. For example, as illustrated in FIG. 2, the light source 114 and/or the optical element 116 can be integrated in the microscope 100. The surgical microscope 100 can include an optical element 118 to direct light from the light source 114 and/or the optical element 116 to the surgical field. The optical element 118 can include a dichroic mirror, a notch filter, a hot mirror, a beamsplitter and/or a cold mirror.

For example, as illustrated in FIG. 3, the light source 114 and/or the optical element 116 can be integrated in the optical block 102. The optical block 102 can be directly or indirectly coupled to the microscope 100 such that the light source 114 and/or the optical element 116 have has a defined optical/optomechanical relationship to the microscope 100. The optical block 128 can be independently manipulable relative to the microscope 100 and/or the procedure eye 104. The optical block 128 can be a hand-held device, a lens holder, a self-stabilized component, or other component. The optical block 128 can be distinct from but coupled to the microscope 100. For example, direct or indirect coupling 138 between the optical block 128 and the surgical microscope 100 can include one or more of a suspension system, a mechanical frame, a protruding arm, a conical structure, a magnetic member, an elastic member, and a plastic member. The optical block 128 can include one or more of the light source 114, the optical element 116, and an optical element 106. In some embodiments, one or more of the light source 114, the optical element 116, and the optical element 106 are omitted from the optical block 128 and included in, for example, the microscope 100 and the instrument 140.

In some embodiments, the light source 114 and/or the optical element 116 do not have a defined optical/optomechanical relationship to one or more components in the surgical imaging system 150, such as the surgical microscope 100. As illustrated in FIG. 4, the light source 114 and/or the optical element 116 can be included in the ophthalmic surgical instrument 140. The instrument 140 can be an endoilluminator, a chandelier, an illuminated infusion cannula, an illuminated vitreoretinal tool, an illuminated cannula, an illuminated laser probe, illuminated scissors, illuminated forceps, etc. For example, the instrument 140 can be maintained separate from and independently positionable relative to the procedure eye 104 and other components the surgical imaging system 150, such as the microscope 100. The instrument 140 can be configured to invasively penetrate a globe of the procedure eye 104. The instrument 140 can be in optical and/or electrical communication with a console 136. The console 136 can include a computing device 122, described in greater detail below. The instrument 140 can include the light source 114 and/or the optical element 116. In some embodiments, the light source 114 and/or the optical element 116 can be omitted from the instrument 140 and included in, for example, the console 136. In such embodiments, light from the light source 114 can be guided to the instrument 140, and the instrument 140 can be used to direct the light to the surgical field.

Referring again to FIG. 1, the method 10 can include, at step 16, determining the emission wavelength(s) associated with the vital stain. The light emitted upon fluorescence of the vital stain can be used to visualize the target biological tissue. The emission wavelength(s) can include a single wavelength, such as a peak emission wavelength, or a range of wavelengths. The range of wavelengths can be centered on a particular wavelength (e.g., all wavelengths within +/−25 nm of the peak emission wavelength). The span of the range can be fixed for all vital stains or variable depending on the particular vital stain used during the surgical procedure.

The emission wavelength(s) can be determined based on the user input identifying the stain used during the ophthalmic surgical procedure. As similarly described above with respect to step 12, the computing device 122 can access a database of vital stains and their corresponding emission wavelength(s), and determine the emission wavelength(s) of the inputted vital stain using the database. In some embodiments, a user input directly identifying the emission wavelength(s) can be received at the user interface 124.

The method 10, at step 18, can include filtering the light from the surgical field using an optical element to allow transmission of light having the emission wavelength(s) and to block transmission of light having the excitation wavelength(s). Light transmitted from the surgical field can be received at one or more components of the surgical imaging system 150. Light transmitted from the surgical field can include emitted light and reflected light. Fluorescent light can be emitted by the vital stain. Light can also be reflected from the surgical field. Controlling the optical element 106 to filter light based on the emission wavelength(s) determined in step 16 can allow for the fluorescent light to be efficiently collected. The target biological tissue to which the stain has been applied can be better visualized when the optical element 106 is controlled to permit transmission of the fluorescent light. Light emitted by auto-fluorescing biological tissue can also be collected and visualized using one or more components of the surgical imaging system 150, such as the microscope optics and/or the image sensor/camera 120.

The optical element 106 can be a spectral filtering element positioned in an optical pathway of the light transmitted from the surgical field. The optical element 106 can be configured to filter the light transmitted from the surgical field based on the emission wavelength(s) of the vital stain. For example, the optical element 106 can block the transmission of light corresponding to the excitation wavelength(s) from the light source 114. The optical element 106 can be a dichroic filter, a diffraction grating, an opto-acoustic tunable filter (AOTF), a liquid crystal tunable filter (LCTF), a linear-variable filter (LVF), a micro-electro-mechanical systems (MEMS) tunable optical filter, interference filter, etc. The optical element 106 can be in communication with the computing device 122. The computing device 122 can provide a control signal to the optical element 106 to filter light based on the emission wavelength(s).

The optical element 106 can be included in one or more components of a surgical imaging system 150 (FIGS. 2-3). For example, the optical element 106 can be integrated in the ophthalmic surgical microscope 100 (FIG. 2) and/or the optical block 128 that is distinct from but coupled to the microscope 100 (FIG. 3).

The optical element 106 can be coupled to the microscope 100, directly or indirectly, such that it has a defined optical/optomechanical relationship to the surgical microscope 100. For example, as illustrated in FIG. 2, the optical element 106 can be integrated in the microscope 100. The optical element 106 can be variously positioned relative to the microscope 100. For example, the element 106 can be disposed in an optical pathway between the procedure eye 104 and the objective lens 112, between the procedure eye 104 and the eyepiece 106, between the procedure eye and the image sensor/camera 120, etc.

For example, as illustrated in FIG. 3, the optical element 106 can be integrated in the optical block 102. The optical block 102 can be directly or indirectly coupled to the microscope 100 such that the optical element 106 has a defined optical/optomechanical relationship to the microscope 100. The optical block 128 can be independently manipulable relative to the microscope 100 and/or the procedure eye 104. The optical block 128 can include one or more of the light source 114, the optical element 116, and the optical element 106. In some embodiments, the optical block 128 includes only the optical element 106. In such embodiments, the optical element 106 can be integrated in a non-contact based optical element. The optical element 106 can be implemented in a manner similar to a non-contact, indirect visualization system (such as a binocular indirect ophthalmomicroscope or BIOM, Zeiss ReSight, Möller-Wedel EIBOS). The optical element 106 can be positioned in the surgical imaging system 150 by one or more of a mechanical coupling to the optical element 116 and/or the light source 114, a mechanical coupling to the optical block 128, a mechanical coupling to the surgical microscope 100, a suspension system, and a lens holder.

The target biological tissue to which the vital stain is applied can be visualized using one or more components of the surgical imaging system 150. For example, fluorescent light emitted from the surgical field can be received at the microscope optics, including the optical components 108 and 110. Thus, the fluorescent target biological tissue can be directly visualized using the microscope optics when the observer 102, such as the surgeon during the ophthalmic surgical procedure, views the surgical field through the eyepiece 106.

For example, fluorescent light emitted from the surgical field can be received at the image sensor/camera 120. The method 10 can include receiving light filtered by the optical element 106 at the image sensor/camera 120. The image sensor/camera 120 can be positioned in the optical pathway of the light transmitted from the surgical field. In some embodiments, the image sensor/camera is part of the microscope 100. In such embodiments, the microscope 100 can include one or more beam splitters 134 to direct at least a portion of the light to the imaging sensor/camera 120. In some embodiments, the image sensor/camera 120 can be a separate component that is not part of the microscope 100 itself, and rather is in communication with the computing device 118, the microscope 100, and/or the optical block 128. For example, the light from the surgical field can be guided to the image sensor/camera 120 by an optical fiber 132 that is coupled to the optical block 128 at a fiber holder 130. The image sensor/camera 120 can include a charge-coupled device (CCD) sensor, a complementary metal-oxide-semiconductor (CMOS) sensor, or other suitable image sensor. For example, a CMOS sensor can be used to implement region-of-interest gain control during image processing to reduce specular reflection from surgical instruments in a selective manner and to enhance visualization of areas of the surgical field with low fluorescence or visibility. The optical element 106 can be positioned in the optical pathway of light transmitted from the surgical field such that light associated with the excitation wavelength(s) are not received at the microscope optics and/or the image sensor/camera 120. The image sensor/camera 120 can include circuitry to generate electrical data and/or image data from the received light.

The image sensor/camera 120 can be in communication with the computing device 122. The image sensor/camera 120 can provide the image data to the computing device 122. In some embodiments, the computing device 122 generates the image data when electrical data is received from the image sensor/camera 120. The method 10 can include processing the light received at the image sensor/camera 120 to generate image data. Processing the image data can include any one or more signal processing steps to prepare the image data for display via the display device 126. For example, processing the image data can include noise reduction, filtering, sharpening, contrast manipulation, etc.

In some embodiments, processing the image data can include image enhancement to facilitate visualization of the target biological tissue to which the vital stain is applied. The method 10 can include determining portions of the image data associated with the emission wavelength(s). For example, the computing device 122 can implement one or more processing steps to identify image data representative of the fluorescent target biological tissue. The method 10 can include modifying a characteristic of the image data to enhance visualization of the target biological tissue in a visual representation display via the display device 126. For example, the computing device 122 can implement one or more processing steps to enhance the image data of the fluorescent target biological tissue displayed by the display device 126. For example, one or more image characteristics, such as intensity, color, contrast, sharpness, boundaries, etc., of the fluorescent target biological tissue can be modified to enhance visualization on the display device 126 compared to direct visualization using the microscope optics. In some embodiments, portions of the image data other than the fluorescent target biological tissue can be modified. The computing device 122 can complete image processing based on one or more user-specified image characteristics received from the user interface 124.

The method 10 can include providing a visual representation of the image data to a display device 126. For example, the computing device 122 can process the image data and provide the processed image data to the display device 126. The display device 126 can be in communication with the computing device 122. The display device 126 can display images of the surgical field captured by the image sensor/camera 120, including the processed image data received from the computing device 122. In some embodiments, the display device 126 is part of the microscope 100. For example, the display device 126 can be a monitor disposed on or coupled to the microscope 100 to allow viewing by the observer 102 and/or other observers. In some embodiments, the display device 126 can be a separate component that is not part of the microscope 100 itself, and rather is in communication with the computing device 122 and the microscope 100. In various embodiments, the display device 126 can be a liquid crystal display (LCD), a light emitting diode liquid crystal display (LED-LCD), a digital micromirror device (DMD), heads up display, near to eye display, and/or other suitable display device. For example, the display device 126 can include transmissive elements (e.g., a backlit LED-LCD) or front-illuminated reflective elements.

The computing device 122 can be in communication, directly or indirectly, with the one or more of the light source 114, the optical element 116, the optical element 106, the image sensor/camera 120, the display device 126, and/or the user interface 124. The computing device 122 can also be in direct or indirect communication with the microscope 100, the optical block 128, and/or the instrument 140. In some embodiments, the computing device 122 is part of the microscope 100 and/or the console 136. In some embodiments, the computing device 122 can be a separate component that is not part of the microscope 100 and/or the console 136. Rather, the computing device 122 can be in communication with one or more components integrated in and/or in communication with the microscope 100, the optical block 128, and/or the instrument 140.

The computing device 118 can include any suitable processor, memory, or processing circuit for executing the steps described herein. For example, the computing device 122 can be configured to generate and provide control signals for the light source 114 and/or the optical element 116 to transmit light to the surgical field based on the excitation wavelength(s). The computing device 122 can be configured to generate and provide a control signal to the optical element 106 to receive light based on the emission wavelength(s). The computing device 122 can be configured to receive electrical data and/or image data from the image sensor/camera 120. The computing device 122 can be configured to generate and/or process the image data. The computing device 122 can be configured to provide processed image data to the display device 126. The computing device 122 can be configured to receive user input from the user interface 120. The computing device 122 can be further configured to perform other steps described herein or necessary to accomplish the steps described herein.

The computing device 122 can be in communication with the user interface 124. In some embodiments, the user interface 104 can be a user-facing component of the computing device 122 such that the user interface 124 is a part of the microscope 100 and/or the console 136. In some embodiments, the user interface 124 is a separate component that is not part of the microscope 100 and/or the console 136. Rather, the user interface 124 can be in communication with the computing device 118 and/or the console 136. The user interface 124 can include input devices or systems, including by way of non-limiting example, a keyboard, a mouse, a joystick, dials, and buttons, among other input devices. The user interface 124 can be a display (including, for example, a touchscreen display) configured to present images or other data (e.g., microscope settings, display settings, etc.) to a user, such as images of surgical field during the surgical procedure. An observer 102 can specify a vital stain, excitation wavelength(s), emission wavelength(s), image characteristics, and/or other inputs for the surgical imaging system 150 via the user interface 104.

In some embodiments, the light source 114 can be configured to transmit light to illuminate the surgical field more generally, as opposed to illumination to excite a fluorescent vital stain. Illumination provided by the light source 114 can enable auto-fluorescence of the target biological tissue (e.g., tissue not stained with the vital stain). The target biological tissue, such the lens, drusen, etc., can be excited when energy from the incident light is absorbed. The target biological tissue can fluoresce as it returns to a ground state. Thus, using the method 10, visualization of surgical targets can be improved even without the use of a vital stain.

In some embodiments, the light source 114 can also provide contrast enhancement for the target biological tissue stained a fluorescent and/or non-fluorescent vital stain. For example, some vital stains can have low visibility when broadband, white light is used illuminate the surgical field. Surgeons can sometimes choose not to use low visibility stains despite having low retinal toxicity because visualization of target biological tissue is difficult. The narrow bandwidth illumination of the present disclosure can provide increased visual contrast for low visibility vital stains. This can increase patient safety by permitting use of safer but lower visibility vital stains that would otherwise have not been used. The contrast enhancement can be implemented independent of or in addition to the fluorescence visualization described herein.

Wavelength(s) of light that increase the visual contrast of the vital stain can be selected to illuminate the surgical field. The wavelength(s) can be selected in a manner similar to selecting the excitation wavelength(s) described above or other suitable manner. In some embodiments, the wavelength(s) that increase contrast are the excitation wavelength(s) associated with the vital stain (e.g., step 12). In some embodiments, a vital stain can stain target biological tissue a color that is markedly different from the color of the vital stain's associated excitation wavelength(s). For example, the wavelength(s) can be separated on the order of, e.g., approximately 100 nm. In other embodiments, the wavelength(s) that improve visual contrast are chosen regardless of the excitation wavelength(s). For example, red light can be chosen to illuminate target biological tissue that is stained green.

Light, such as narrow bandwidth light, can be transmitted to the surgical field (e.g., step 14) based on the contrast-maximizing wavelength(s). Light can be transmitted to the surgical field in a similar manner as described above. For example, a tunable light source can be controlled to transmit light having the contrast-increasing wavelength(s). For example, light from a light source can be filtered with an optical element positioned in an optical pathway of the light transmitted by the light source such that the transmitted light has the contrast-increasing wavelength(s). In some embodiments, the surgical field can be illuminated with additional light to provide situational awareness for the surgeon. For example, low intensity, uniform, and/or broadband light can be provided in addition to the narrow bandwidth light. The uniform light can be provided from a light source included in the microscope 100, the instrument 140, and/or other component of the surgical imaging system 150.

The reflection of light transmitted to the surgical field (e.g., including the narrow bandwidth light) can be collected and visualized using one or more components of the surgical imaging system 150, such as the microscope optics and/or the image sensor/camera 120. The target biological tissue stained by the vital stain can be accentuated compared to other portions of the surgical field. The contrast of the target biological tissue can be enhanced because the color of the stained target biological tissue is markedly different from the color of the narrow bandwidth light transmitted to the surgical field. For example, a vital stain that stains target biological tissue blue can have an absorption peak in the yellow wavelength range. The light source 114 can illuminate the surgical field with narrow bandwidth, yellow light. The blue-stained target biological tissue can be more readily identified with the surgical field at least partially illuminated with the narrow bandwidth, yellow light.

In embodiments in which in light reflected from the surgical field is received at the image sensor/camera 120, the computing device 122 can implement one or more processing steps to enhance the image data of the stained target biological tissue displayed by the display device 126, as similarly described above. For example, one or more image characteristics, such as intensity, color, contrast, sharpness, boundaries, etc., of the stained target biological tissue can be modified to enhance visualization on the display device 126 compared to direct visualization using the microscope optics. In some embodiments, portions of the image data other than the stained target biological tissue can be modified to increase the contrast between the vital stain and the surgical field at large.

The microscope 100 discussed herein can be a monocular or binocular microscope. It is understood that the microscope 100 can include one eyepiece for each eye of one or more observers 102 (e.g., two eyepieces each for a surgeon and an assistant). The teaching of the present disclosure can be implemented such that fluorescent target biological tissue is viewable through one or more eyepieces. The microscope 100 can be a compound, stereo, or digital microscope. The teaching of the present disclosure can be implemented in one or more optical paths of the microscope 100. For example, the optical element 106 can be controlled to filter light based on the emission wavelength(s) in the single optical pathway between the observer 102 and the surgical field in a compound or digital microscope. For example, the optical element 106 can be controlled to filter light based the emission wavelength(s) in the two optical paths between the observer 102 and surgical field in a stereo microscope. In some embodiments, the optical element 106 is provided for each optical pathway associated the eyes of the observer 102. In some embodiments, the separate image data is combined before being provided to the microscope optics and/or the display device 126. In some embodiments, image data is separately generated for each of multiple observers 102 (e.g., for one eye of each observer, for both eyes of each observer, separately for each eye of each observer, etc.).

Embodiments as described herein can provide devices, systems, and methods that facilitate visualization of target biological tissue by efficiently exciting a vital stain to fluoresce and controlling the collection the fluorescent light. Embodiments described herein also facilitate visualization by selectively illuminating the surgical field with narrow bandwidth light that increases the contrast of stained target biological tissue. While one or more specific stains or categories of stains have been referred to herein as examples, it is understood that the teachings of the present disclosure can be applied to any stain, dye, and/or other coloring-agent. Similarly, while surgical procedures have been referred to herein as examples, it is understood that the teachings of the present disclosure can be applied during any ophthalmic procedures, including during in-clinic and/or in-office diagnosis, treatment, check-up, follow-up, etc. The examples provided above are exemplary only and are not intended to be limiting. One skilled in the art may readily devise other systems consistent with the disclosed embodiments which are intended to be within the scope of this disclosure. As such, the application is limited only by the following claims.

1-19. (canceled)

20. An ophthalmic surgical imaging system, comprising:
a light source controllable to transmit light having an excitation wavelength associated with a vital stain disposed in a surgical field;
a first optical element disposed in an optical pathway of light transmitted from a surgical field, the first optical element being controllable to selectively filter light received from the surgical field to allow the transmission of light having an emission wavelength associated with the vital stain and to block the transmission of light having the excitation wavelength;
an image sensor disposed in the optical pathway of light transmitted from the surgical field, the image sensor being configured to receive the filtered light having the emission wavelength from the first optical element;
a computing device in communication with the image sensor and configured to process the received light to generate image data including modifying a characteristic of the image data to enhance visualization of the target biological tissue in a visual representation; and
a display device in communication with computing device and configured to display the visual representation.

21. The system of claim 20, further comprising:
a second optical element disposed in an optical pathway of the light transmitted by the light source, the first optical element being controllable to selectively filter the light such that light having the excitation wavelength is transmitted to the surgical field.

22. A method of ophthalmic imaging, comprising:
determining a wavelength of light that increases the visual contrast of a vital stain disposed in a procedure eye;
transmitting light having the determined wavelength to the procedure eye including at least one of:
controlling a tunable light source to transmit the light having the determined wavelength; and
filtering light from a light source with an optical element positioned in an optical pathway of light transmitted by the light source to transmit the light having the determined wavelength; and
receiving a reflection of the light transmitted to the procedure eye such that target biological tissue stained by the vital stain is accentuated compared to other portions of the procedure eye.

23. The method of claim 22, further comprising:
receiving light reflected from the procedure eye at an image sensor;
processing the received light to generate image data; and
outputting a visual representation of the image data to a display device.

24. The method of claim 23, further comprising:
determining portions of the image data associated with the vital stain; and
modifying a characteristic of the image data to enhance visualization of target biological tissue in the visual representation.

25. The method of claim 22, further comprising at least one of:
intravitreally injecting the vital stain to stain the target biological tissue in the procedure eye;
intravenously delivering the vital stain to the target biological tissue in the procedure eye; and
delivering the vital stain to the procedure eye such that the vital stain is infused into the target biological tissue.

26. A system for imaging in an ophthalmic surgical procedure, comprising:
a light source configured to transmit light; and
a computing device configured to:
receive an input identifying a vital stain;
determine an excitation wavelength of light associated with the vital stain disposed in a surgical field;
control the light source to transmit light having the determined excitation wavelength to the surgical field;
determine an emission wavelength of light associated with the vital stain; and
control a first optical element to filter light from the surgical field to allow transmission of light having the determined emission wavelength and to block transmission of light having the determined excitation wavelength.

27. The system of claim 26, wherein the vital stain is intravitreally injected to stain a target biological tissue in the surgical field.

28. The system of claim 26, wherein the vital stain is intravenously delivered to a target biological tissue in the surgical field.

29. The system of claim 26, wherein the vital stain is infused into a target biological tissue.

30. The system of claim 26, wherein:
the light source comprises a tunable light source; and
the computing device is configured to control the tunable light source to transmit the light having the excitation wavelength.

31. The system of claim 26, wherein the computing device is configured to control the light source by:
filtering light from a light source with a second optical element positioned in an optical pathway of light transmitted by the light source to transmit the light having the excitation wavelength.

32. The system of claim 31, wherein:
at least one of the light source and the second optical element are integrated in an ophthalmic surgical instrument.

33. The system of claim 32, wherein the ophthalmic surgical instrument is at least one of:
an endoilluminator, a chandelier, an illuminated infusion cannula, illuminated vitreoretinal tool, an illuminated cannula, an illuminated laser probe, illuminated scissors, and illuminated forceps.

34. The system of claim 31, wherein:
at least one of the light source and the second optical element are disposed in a defined optical relationship to an ophthalmic surgical microscope positioned in an optical pathway of light transmitted from the surgical field.

35. The system of claim 34, wherein:
at least one of the light source and the second optical element are integrated in the ophthalmic surgical microscope.

36. The system of claim 34, wherein:
at least one of the light source and the second optical element is coupled to the ophthalmic surgical microscope by at least one of a suspension system, a mechanical frame, a protruding arm, a conical structure, a magnetic member, an elastic member, and a plastic member.

37. The system of claim 36, wherein:
at least one of the light source and the second optical element is integrated into an optical block.

38. The system of claim 26, wherein:
the first optical element is disposed in a defined optical relationship to an ophthalmic surgical microscope positioned in an optical pathway of light transmitted from the surgical field.

39. The system of claim 37, wherein:
the first optical element is integrated in the ophthalmic surgical microscope.

40. The system of claim 38, wherein:
the first optical element is coupled to the ophthalmic surgical microscope by at least one of a suspension system, a mechanical frame, a protruding arm, a conical structure, a magnetic member, an elastic member, and a plastic member.

41. The system of claim 40, wherein:
the first optical element is integrated into an optical block.

42. The system of claim 38, wherein:
the first optical element is integrated in a non-contact optical element, positioned by at least one of
a mechanical coupling to the ophthalmic surgical microscope;
a suspension system; and
a lens holder.

43. The system of claim 26, further comprising an image sensor configured to:
receive light filtered by the first optical element at an image sensor;
process the received light to generate image data; and
output a visual representation of the image data to a display device.

44. The system of claim 43, wherein the computing device is further configured to:
determine portions of the image data associated with the emission wavelength; and
modify a characteristic of the image data to enhance visualization of target biological tissue in the visual representation.
